(19) United States
(12) Patent Application Publication
(10) Pub. No.: US 2021/0355065 A1
(43) Pub. Date: Nov. 18, 2021

(54) CB-0406 tromethamine salt

(71) Applicants: CymaBay Therapeutics, Inc., Newark, CA (US); DiaTex, Inc., San Antonio, TX (US)

(72) Inventors: Stephan X.M. Boerrigter, West Lafayette, IN (US); Charles A. McWherter, Oakland, CA (US); Robert L. Martin, San Ramon, CA (US); Jennifer L. Nelson, Kokomo, IN (US); Jiangao Song, Sunnyvale, CA (US)

(73) Assignees: CymaBay Therapeutics, Inc., Newark, CA (US); DiaTex, Inc., San Antonio, TX (US)

(21) Appl. No.: 17/318,681

(22) Filed: May 12, 2021

Related U.S. Application Data
(60) Provisional application No. 63/026,243, filed on May 18, 2020.

(51) Int. Cl. C07C 59/72; C07C 215/40

(52) U.S. Cl. CPC C07C 59/72; C07B 2200/13; C07C 215/40

(57) ABSTRACT

CB-0406 tromethamine salt, especially in crystalline form and as an ansolvate, methods of preparing it, compositions containing it, and its pharmaceutical uses.

### CROSS-REFERENCE TO RELATED APPLICATION

This application claims the benefit under 35 USC 119(e) of Application No. 63/026,243, “CB-0406 tromethamine salt”, filed 18 May 2020, the entire content of which is incorporated into this application by reference.

### FIELD OF THE INVENTION

This invention relates to the tromethamine salt of CB-0406.

### DESCRIPTION OF THE RELATED ART

CB-0406

CB-0406 is the compound having the IUPAC name of (2R)-2-(4-chlorophenyl)-2-[3-(trifluoromethyl)phenoxy]acetic acid, sometimes also given as (R)-2-(4-chlorophenyl)-2-[3-(trifluoromethyl)phenoxy]acetic acid. Other names that are or have been used for CB-0406 are (−)-CPTA, and arhalofenic acid or arhalofenate acid, since it is the underlying acid of the compound arhalofenate [INN/USAN; (−)-2-(acetylamino)ethyl (2R)-2-(4-chlorophenyl)-2-[3-(trifluoromethyl)-phenoxy]acetate; MBX-0102, CB-0102]. CB-0406 is the active metabolite of arhalofenate [see, for example, McWherter et al., “Arhalofenate acid inhibits monosodium urate crystal-induced inflammatory responses through activation of AMP-activated protein kinase (AMPK) signaling”, Arthritis Res. Ther., vol. 20, 204 (2018), https://doi.org/10.1186/s13075-018-1699-4]. U.S. Pat. No. 6,262,118 discloses the use of arhalofenate, CB-0406, and related compounds for the treatment of insulin resistance, type 2 diabetes, and hyperlipidemia, and U.S. Pat. No. 6,613,802 adds the treatment of hyperuricemia to that list. Those patents explain that arhalofenate and related compounds avoid certain drug-drug interactions seen with the racemate, such as with sulfonylureas, NSAIDs, and the anticoagulant warfarin, an interaction believed to be mediated by inhibition of certain cytochrome P450 enzymes, particularly CYP 2C9; and demonstrate that CB-0406 was approximately 20-fold less active as an inhibitor of CYP 2C9 than its (S)-enantiomer in the tolbutamide hydroxylation assay. U.S. Pat. Nos. 9,023,856 and 9,060,987, for example, disclose the treatment of hyperuricemia and gout, including gout flares, with arhalofenate, CB-0406 and its salts, and related compounds.

U.S. Pat. No. 6,262,118 discloses a synthesis of CB-0406 by resolution of its racemate with (−)-cinchonidine, thereby isolating the (−)-cinchonidine salt of CB-0406. U.S. Pat. No. 7,199,259 discloses a synthesis of CB-0406 by resolution with various agents, in particular (1R,2R)-2-amino-1-(4-nitrophenyl)propane-1,3-diol [CAF D base], thereby isolating the CAF D base salt of CB-0406; and U.S. Pat. No. 7,432,394 discloses a synthesis of CB-0406 by resolution of its racemate with a variety of chiral aralkylamines, in particular (S)-1-(2-naphthyl)ethylamine, thereby isolating the (S)-1-(2-naphthyl)ethylamine salt of CB-0406. Others, e.g. U.S. Pat. Nos. 7,714,131 and 8,541,614, disclose stereoselective syntheses, typically considering CB-0406 as an intermediate to arhalofenate.

U.S. Pat. No. 9,023,856, for example, says the following about salts of CB-0406:
“Pharmaceutically acceptable salt” includes pharmaceutically acceptable acid addition salts and pharmaceutically acceptable base addition salts and includes both solvated and unsolvated forms. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.
“Pharmaceutically acceptable base addition salt” refers to salts prepared from the addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylamino-ethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

The disclosures of the documents referred to in this application are incorporated into this application by reference.

### SUMMARY OF THE INVENTION

In a first aspect, this invention is CB-0406 tromethamine salt. In particular, this aspect is crystalline CB-0406 tromethamine salt, CB-0406 tromethamine salt ansolvate, and especially crystalline CB-0406 tromethamine salt ansolvate.

In a second aspect, this invention is methods of preparing the CB-0406 tromethamine salt of the first aspect of this invention.

In a third aspect, this invention is pharmaceutical compositions, especially oral pharmaceutical compositions, containing the CB-0406 tromethamine salt of the first aspect of this invention.

In a fourth aspect, this invention is pharmaceutical uses of the CB-0406 tromethamine salt of the first aspect of this invention in the treatment of conditions for which arhalofenate, or CB-0406 and its salts, are indicated.

Preferred embodiments of this invention are characterized by the specification and by the features of Claims 1 to 16 of this application as filed.

### BRIEF DESCRIPTION OF THE DRAWINGS

FIG. 1 is a differential scanning calorimetry (DSC) thermogram of CB-0406 tromethamine salt.

FIG. 2 is a thermogravimetric analysis (TGA) thermogram of CB-0406 tromethamine salt.

FIG. 3 is a dynamic vapor sorption (DVS) thermogram of CB-0406 tromethamine salt.

FIG. 4 is an X-ray powder diffraction (XRPD) pattern of CB-0406 tromethamine salt.

### DETAILED DESCRIPTION OF THE INVENTION

### Definitions

“CB-0406” is described in the section “CB-0406” in the DESCRIPTION OF THE

### RELATED ART

“Tromethamine” [INN/USAN] has the IUPAC name 2-amino-2-hydroxymethyl-1,3-propanediol. It is also referred to as trometamol, tromethane, trimethylolaminomethane, tris(hydroxymethyl)aminomethane, trisamine, tris, and THAM.

“CB-0406 tromethamine salt” is the 1:1 salt formed between (2R)-2-(4-chlorophenyl)-2-[3-(trifluoromethyl)phenoxy]acetic acid and 2-amino-2-(hydroxymethyl)propane-1,3-diol. It may be named 1,3-dihydroxy-2-(hydroxymethyl)propan-2-aminium (2R)-2-(4-chlorophenyl)-2-[3-(trifluoromethyl)phenoxy]acetate. “Crystalline CB-0406 tromethamine salt” is a crystalline solid form of CB-0406 tromethamine salt. The “ansolvate” of CB-0406 tromethamine salt is a form of CB-0406 tromethamine salt that is free of solvents associated with the salt, including water; but bulk material may contain small amounts of one or more solvents, such as the solvents used in its synthesis. The “crystalline ansolvate” of CB-0406 tromethamine salt is a crystalline form of CB-0406 tromethamine salt that is free of solvents of crystallization associated with the salt, including water; but bulk material may contain small amounts of one or more solvents, such as the solvents used in its synthesis or crystallization.

“Characterization” refers to obtaining data that may be used to identify a solid form of a compound; for example, whether the solid form is amorphous or crystalline and whether it is unsolvated or solvated. The process by which solid forms are characterized involves analyzing data collected on the forms to allow a person of ordinary skill in the art to distinguish one solid form from other solid forms containing the same material. Chemical identity of solid forms can often be determined with solution-state techniques such as ¹³C nuclear magnetic resonance (NMR) spectroscopy or ¹H NMR. While these may help identify a material, and a solvent molecule for a solvate, such solution-state techniques themselves do not provide information about the solid state. There are, however, solid-state analytical techniques that can be used to provide information about solid-state structure and differentiate among solid forms such as polymorphs, including single crystal X-ray diffraction, XRPD, solid state NMR, infrared and Raman spectroscopy, and thermal techniques such as DSC, TGA, melting point, and hot-stage microscopy.

An XRPD pattern is an x-y graph with diffraction angle 2θ (typically in degrees, °) on the x-axis and intensity on the y-axis. The peaks within this pattern may be used to characterize a crystalline solid form. As with any data measurement, there is variability in XRPD data. The data are frequently represented solely by the diffraction angle of the peaks rather than including the intensity of the peaks because peak intensity can be particularly sensitive to sample preparation, for example, because of particle morphology and size, moisture content, solvent content, and preferred orientation effects, so samples of the same material prepared under different conditions may yield slightly different XRPD patterns; and this variability is usually greater than the variability in diffraction angles. Diffraction angle variability may also be sensitive to sample preparation. Other, but less significant, sources of diffraction angle variability come from instrument parameters and processing of the raw X-ray data: different instruments operate using different parameters and these may lead to slightly different XRPD patterns even from the same solid form, and similarly different software packages process X-ray data differently and this also leads to variability. These and other sources of variability are known to those of ordinary skill in the pharmaceutical arts. Due to such sources of variability, it is usual to assign a variability of ±0.2° to diffraction angles (2θ) in XRPD patterns, especially when using those angles for characterization of a solid form.

To characterize a solid form of a compound a person of ordinary skill in the art may, for example, collect XRPD data on solid forms of the compound and compare the XRPD peaks of the forms. When only two solid forms, I and II, are compared and the Form I XRPD pattern shows a peak at an angle where no peaks appear in the Form II XRPD pattern, then for that compound that peak distinguishes Form I from Form II and further acts to characterize Form I. The collection of peaks that distinguish Form I from the other known forms is a collection of peaks that may be used to characterize Form I. Additional peaks could also be used, but are not necessary, to characterize the form, up to and including an entire XRPD pattern; however, a subset of that data may, and typically is, used to characterize the form. A person of ordinary skill in the art will recognize that there are often multiple ways, including multiple ways using the same technique, to characterize solid forms.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

CB-0406 tromethamine salt has been characterized using DSC, TGA, XRPD, hot stage microscopy, and solution ¹H NMR. CB-0406 tromethamine salt has also been tested by relative humidity stressing, and its solubility measured in simulated intestinal fluid without pancreatin.

Preparation of CB-0406 Tromethamine Salt

Preparation of CB-0406 tromethamine salt by slurrying. CB-0406 (66.5 mg) and one molar equivalent of tromethamine (24.0 mg) were added to 83/17 v/v cyclohexane/ethanol (1.2 mL). The mixture was heated while stirring at approximately 70° C. in an oil bath on a hot plate. After approximately 15 minutes, a few solid particles remained. The solution was hot filtered into a pre-warmed vial, and the vial was placed into the oil bath at approximately 70° C. The hot plate was turned off, and the sample was slow cooled to ambient temperature. At ambient temperature, oily and aqueous layers were observed. The vial was removed from the oil bath and placed uncapped under a nitrogen stream for evaporation. A sticky film resulted and was dissolved in 0.3 mL of ethyl acetate. The solution was uncapped and placed under a nitrogen gas flow for evaporation. A viscous, sticky film resulted and was placed in a vacuum oven at ambient temperature overnight. A glass resulted. The vial was purged with nitrogen gas and the glass was dissolved in approximately 0.5 mL of anhydrous methyl tert-butyl ether (MTBE). The solution was evaporated under a nitrogen stream. A gel-like material resulted, and approximately 0.5 mL of anhydrous heptane was added. The solution was sonicated for approximately 1 minute and solids precipitated. The mixture was stirred at ambient temperature for 1 day. The solids were isolated by vacuum filtration, and the wet cake was washed twice with approximately 0.5 mL of anhydrous MTBE to give CB-0406 tromethamine salt.

Preparation of CB-0406 tromethamine salt in solution. CB-0406 (3.30 g) was added to a solution of tromethamine (1.21 g) in MTBE (25 mL). The mixture was stirred at 55° C. (oil bath temperature) for 2 hours. A small amount of solid was removed by filtration. Solvent was removed by rotary evaporation, and the residue was dried under vacuum pump overnight. Heptane (100 mL) was added to the flask, and mixture was stirred overnight. The mixture was rotated in a rotary evaporator at 65-75° C. for 2 hours, then was cooled down to ambient temperature. Filtration and drying under vacuum gave 4.17 g (92%) of solid CB-0406 tromethamine salt, m.p. (121-122) ° C.

Preparation of CB-0406 tromethamine salt in solution. Tromethamine (1.21 g) was added to a solution of CB-0406 (3.30 g) in isopropyl alcohol (25 mL). The mixture was stirred at 55° C. (oil bath temperature) for 80 min. Solvent was removed by rotary evaporation. Heptane (25 mL) was added and removed by rotary evaporation. Heptane (25 mL) was added and rotated in a rotary evaporator at 65-75° C. for 2 hours, then was cooled down to ambient temperature. Filtration and drying under vacuum gave 4.43 g (98%) of solid CB-0406 tromethamine salt, m.p. (121-122) ° C.

Preparation of CB-0406 tromethamine salt in solution. Tromethamine (213.9 g, 1.766 mol, 1.0 eq) was added to a solution of CB-0406 (584 g, 1.766 mol, 1.0 eq) in isopropyl alcohol (4.08 L). The mixture was stirred at −70° C. for 3 hours, giving a clear solution, then cooled to ambient temperature and filtered. The filtrate was dried over anhydrous sodium sulfate and concentrated under reduced pressure to an oil. Heptane (4.8 L×2) was added and removed by rotary evaporation to give CB-0406 tromethamine salt crude product (900 g) as a pale yellow foam. This was stirred in heptane (4.0 L) at 65-75° C. for 2 hours, during which time some white solid slowly precipitated, then at ambient temperature overnight. The mixture was filtered and the filter cake washed with heptane (500 mL×3), and dried under reduced pressure to give CB-0406 tromethamine salt (787 g, 98.7%) as a white solid, m.p. (118.8-120.0) ° C.

Characterization of CB-0406 Tromethamine Salt

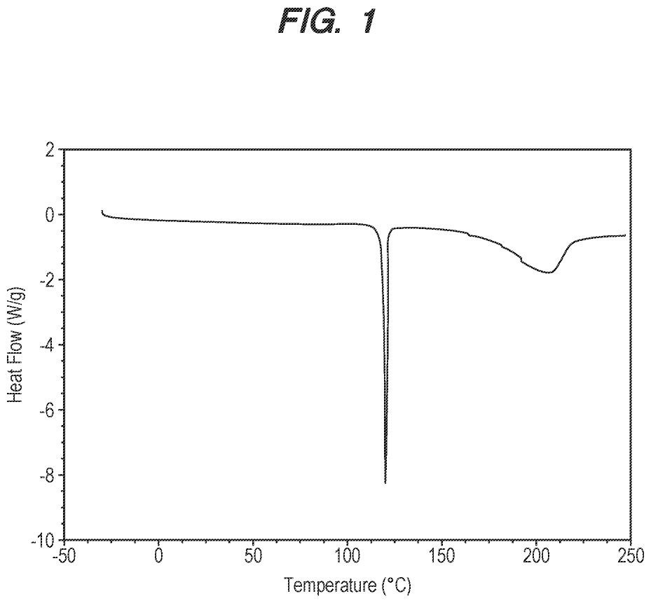

A DSC analysis of CB-0406 tromethamine salt was performed using a TA Instruments Q2000 differential scanning calorimeter. Temperature calibration was performed using NIST-traceable indium metal. The sample, 1.27 mg, was placed into an aluminum DSC pan, covered with a lid which was crimped at the beginning of the run, and the weight was accurately recorded. A weighed aluminum pan configured as the sample pan was placed on the reference side of the cell. The sample cell was heated from −30° C. to 250° C. at 10° C./minute. As shown in FIG. 1, DSC showed a steep initial endotherm with onset at about 115° C. and peak (95.3 J/g) at 119.8° C., with a broad endotherm peaking at around 210° C. The variability of DSC data is affected by sample preparation and particularly by heating rate.

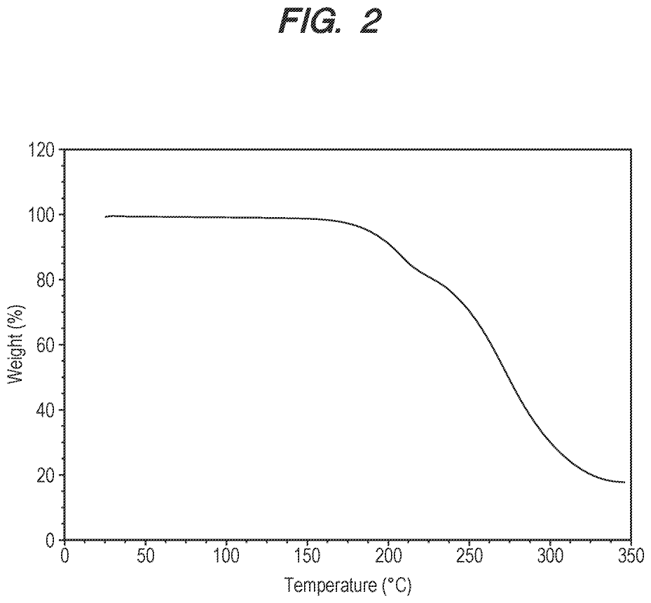

A TG analysis of CB-0406 tromethamine salt was performed using a TA Instruments 2950 thermogravimetric analyzer. Temperature calibration was performed using nickel and Alumel™ The sample, 4.825 mg, was placed in an aluminum pan and inserted into the TG furnace. The furnace was heated under a nitrogen purge. The sample cell was heated from ambient temperature to 350° C. at 10° C./minute. As shown in FIG. 2, TGA showed a negligible loss in weight (0.6%) between 75° C. and 133° C., and a steepening loss starting at about 175° C. As with DSC data, the variability of TGA data is affected by sample preparation and particularly by heating rate.

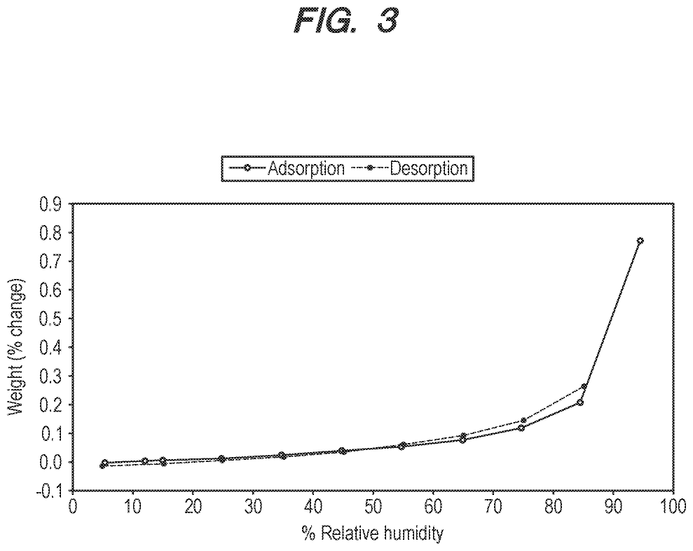

A DVS analysis of CB-0406 tromethamine salt was performed using a VTI SGA-100 Vapor Sorption Analyzer. The sample was not dried prior to analysis, and data were not corrected for the initial moisture content of the sample. Equilibrium criteria used for analysis were less than 0.010% weight change in 5 minutes, with a maximum equilibration time of 3 hours if the weight criterion was not met. Data were not corrected for the initial moisture content of the sample. Sodium chloride and polyvinylpyrrolidone were used as calibration standards. As shown in FIG. 3, DVS showed a net weight gain of less than 0.8% (approximately equal to 0.2 equivalents of water) at 95% relative humidity relative to the weight at 5% relative humidity, and this weight gain was essentially completely reversible.

The XRPD pattern of CB-0406 tromethamine salt was collected with a PANalytical X'Pert PRO MPD diffractometer using an incident beam of Cu radiation produced using an Optix long, fine-focus source at 45 kV and 40 mA, with a 0.5° divergence slit before the mirror. An elliptically graded multilayer mirror was used to focus Cu Kα X-rays through the specimen and onto the detector. Prior to the analysis, a silicon specimen (NIST SRM 640d) was analyzed to verify the observed position of the Si 111 peak is consistent with the NIST-certified position. A specimen of the sample was sandwiched between 3 m thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The scan range was (1.00-39.99)° 2θ, with a scan speed of 3.3°/minute (step size 0.017° 2θ).

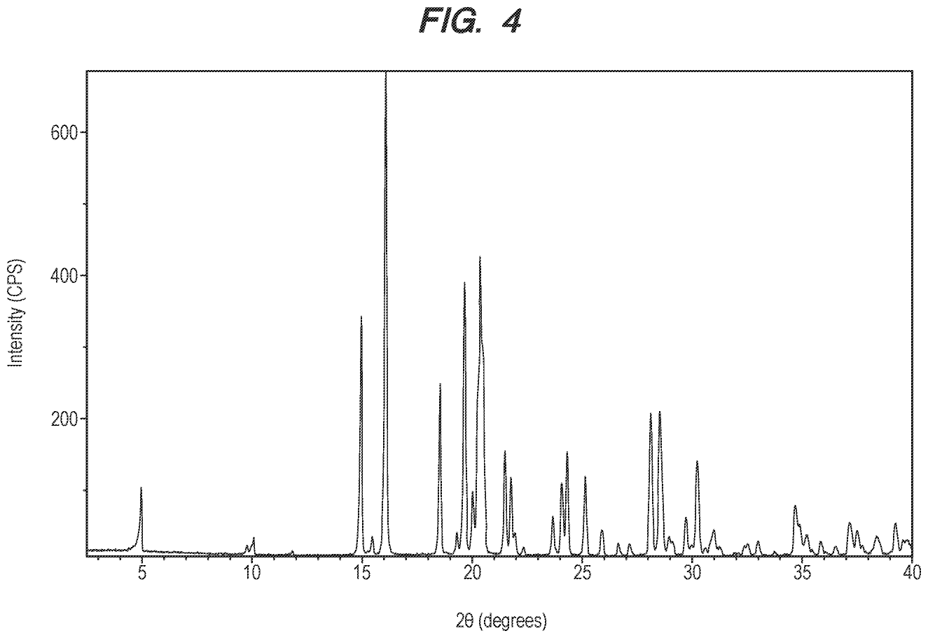

The XRPD pattern is shown in FIG. 4. The location of the peaks along the horizontal axis was automatically determined using proprietary software (PatternMatch v.3.0.4) and rounded to two decimal places. Peaks in diffraction intensity, with the intensity in parentheses as a percentage of the maximum recorded intensity (the intensity of the peak at 16.09°), were determined from the XRPD pattern of FIG. 4 at 4.98° (15), 9.79° (3), 10.08° (5), 11.83° (2), 14.97° (50), 15.48° (5), 16.09° (100), 18.55° (36), 19.32° (6), 19.67° (58), 20.04° (14), 20.37° (63), 21.49° (23), 21.78° (17), 21.94° (6), 22.33° (3), 23.66° (9), 24.08° (16), 24.32° (23), 25.14° (18), 25.89° (7), 26.64° (4), 27.17° (4), 28.13° (30), 28.54° (30), 28.96° (5), and 29.71° (9).

Prominent peaks used for characterization may be selected from this list, such as those at having intensities greater than 15% of the maximum recorded intensity (the intensity of the peak at 16.09°), i.e., peaks at 5.0°, 15.0°, 16.1°, 18.6°, 19.7°, 20.0°, 20.4°, 21.5°, 21.8°, 24.1°, 24.3°, 25.1°, 28.1°, and 28.5°; figures here are rounded to only one decimal place because of the assumed ±0.2° variability in 2θ. Of these, low diffraction angle and high intensity peaks are of greatest interest, such as the peaks at 5.0°, 15.0°, 16.1°, 18.6°, 19.7°, 20.4°, 28.1°, and 28.5° 20. An XRPD pattern “substantially similar” to the pattern shown in FIG. 4 will exhibit at least five of the peaks listed in the preceding sentence to within ±0.2° in 2θ, though not necessarily at the intensities listed in the previous paragraph.

Indexing and subsequent Pawley refinement (Pawley, G. S., “Unit cell refinement from powder diffraction scans”, J. Appl. Cryst., 14, 357-361 (1981)) provides the most accurate determination of unit cell volume and cell parameters from XRPD data. These computations were performed using TOPAS 4.2 (Topas 4.2, 2009, Bruker AXS GmbH, Karlsruhe, Germany). The background was modeled using a 3rd order Chebychev polynomial and increased where needed. Peak shape was modeled using Lorentzian crystallite size broadening. Absorption and 2θ corrections were made for sample thickness and displacement, respectively. Peak positions were allowed to vary by fitting the unit cell parameters. Whole pattern Pawley refinement was performed on all parameters simultaneously to a convergence of 0.001 in χ². Pawley refined cell (Rwp=5.07%): C2, a=18.3751 Å, b=6.2514 Å, c=18.0386 Å, β=101.328°; volume=2031.71 Å³ (predicted volume for a non-solvated 1:1 salt in this space group: 2038.64 Å³).

Hot stage microscopy of CB-0406 tromethamine salt was performed using a Linkam hot stage (FTIR 600) mounted on a Leica DM LP microscope equipped with a SPOT Insight™ color digital camera. Temperature calibrations were performed using USP melting point standards. The sample was placed on a cover glass, and a second cover glass was placed on top of the sample. As the stage was heated at 10° C./minute, the sample was visually observed using a 20× objective with crossed polarizers and a first order red compensator. No changes were observed below 103.2° C., and melting was observed at 120.2° C. and was complete (last crystal melted) by 121.3° C.

A solution ¹H NMR spectrum of CB-0406 tromethamine salt was acquired with a Varian ^{UNITY}INOVA-400 spectrometer. The sample was prepared by dissolving a small amount of CB-0406 tromethamine salt, prepared by slurrying CB-0406 and tromethamine in heptane as described previously, in DMSO-d₆ containing tetramethylsilane. The spectrum of CB-0406 tromethamine salt was consistent with the presence of deprotonated CB-0406 to tromethamine in about a 1:1 ratio, with a trace of heptane.

Relative humidity (RH) stressing of CB-0406 tromethamine salt was performed by placing solid CB-0406 tromethamine salt in an RH chamber containing a saturated aqueous solution of sodium chloride (˜75% RH), potassium chloride (˜84% RH, RT/-82% RH, 40° C.), sodium bromide (˜58% RH, RT), or potassium sulfate (˜97% RH, RT/-96% RH, 40° C.), with excess salt present. The chamber was sealed and left at ambient temperature or placed in an oven at elevated temperature. At 75% RH and 41° C., the CB-0406 tromethamine salt was observed as a free-flowing solid at 7 and 9 days, and XRPD analysis was consistent with CB-0406 tromethamine salt at 18 and 46 days, though some increase in low angle scattering background was seen.

CB-0406 tromethamine salt was determined to have a solubility >200 mg/mL in simulated intestinal fluid without pancreatin.

Pharmaceutical Formulations

CB-0406 tromethamine salt is expected to be of pharmaceutical utility because of its ability to be produced in crystalline form, with a higher melting point than crystalline CB-0406 (i.e. ˜119° C. for CB-0406 tromethamine salt, −99° C. for CB-0406), and with good stability to thermal and relative humidity stress. It also has high solubility in simulated intestinal fluid (at least ˜60-fold greater than that of CB-0406), leading to expected high oral bioavailability. Though it is expected to be useful in formulations other than oral formulations because of its desirable pharmaceutical properties, it is expected to be of particular value in oral formulations. Suitable formulations for various methods of administration may be found, for example, in “Remington: The Science and Practice of Pharmacy”, 20th ed., Gennaro, ed., Lippincott Williams & Wilkins, Philadelphia, Pa., U.S.A. Because CB-0406 tromethamine salt is soluble and therefore orally available, typical formulations will be oral, and typical dosage forms will be tablets or capsules for oral administration. In addition to an effective amount of the CB-0406 tromethamine salt, the compositions may contain one or more suitable pharmaceutically acceptable excipients, including fillers, stabilizers such as antioxidants, disintegrating agents, and processing aids such as binders, glidants, and lubricants, which facilitate processing of the CB-0406 tromethamine salt into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered. For solid compositions, conventional excipients include, for example, pharmaceutical grades of mannitol, lactose, starch, magnesium stearate, sodium saccharin, talc, cellulose, glucose, sucrose, magnesium carbonate, and the like.

Pharmaceutical Uses

CB-0406 tromethamine salt, as a salt of CB-0406, is expected to be pharmaceutically useful in the treatment of all conditions for which arhalofenate, or CB-0406 and its salts, are indicated. It is thus expected to be useful for the treatment of insulin resistance, type 2 diabetes, hyperlipidemia, and hyperuricemia, as described for example in U.S. Pat. Nos. 6,262,118 and 6,613,802; and for the treatment of hyperuricemia and gout, including gout flares, as described for example in U.S. Pat. Nos. 9,023,856 and 9,060,987.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A compound that is 1,3-dihydroxy-2-(hydroxymethyl)propan-2-aminium (2R)-2-(4-chlorophenyl)-2-[3-(trifluoromethyl)phenoxy]acetate.

2. The compound of claim 1 in crystalline form.

3.-16. (canceled)

17. The compound of claim 1 that is an ansolvate.

18. The compound of claim 17 in crystalline form.

19. The compound of claim 1 characterized by at least one of (a), (b), (c), or (d):
(a) an endothermic peak at (120±2) ° C. as measured by differential scanning calorimetry;
(b) a substantial absence of weight loss below 133° C. as measured by thermogravimetric analysis;
(c) a melting point of (120±2) ° C. as measured by hot stage microscopy;
(d) at least one X-ray powder diffraction peak (Cu Kα radiation) selected from 5.0°, 15.0°, 16.1°, 18.6°, 19.7°, 20.4°, 28.1°, or 28.5° (each ±0.2°) 2θ.

20. The compound of claim 19 characterized by an endothermic peak at (120±2) ° C. as measured by differential scanning calorimetry.

21. The compound of claim 20 characterized by a substantial absence of thermal events at temperatures below the endothermic peak at (120±2) ° C. as measured by differential scanning calorimetry.

22. The compound of claim 19 characterized by a substantial absence of weight loss below 133° C. as measured by thermogravimetric analysis.

23. The compound of claim 19 characterized by a melting point of (120±2) ° C. as measured by hot stage microscopy.

24. The compound of claim 19 characterized by at least one X-ray powder diffraction peak (Cu Kα radiation) selected from 5.0°, 15.0°, 16.1°, 18.6°, 19.7°, 20.4°, 28.1°, or 28.5° (each ±0.2°) 2θ.

25. The compound of claim 24 characterized by at least two X-ray powder diffraction peaks (Cu Kα radiation) selected from 5.0°, 15.0°, 16.1°, 18.6°, 19.7°, 20.4°, 28.1°, or 28.5° (each ±0.2°) 2θ.

26. The compound of claim 25 characterized by at least three X-ray powder diffraction peaks (Cu Kα radiation) selected from 5.0°, 15.0°, 16.1°, 18.6°, 19.7°, 20.4°, 28.10, or 28.5° (each ±0.2°) 2θ.

27. The compound of claim 24 characterized by an X-ray powder diffraction peak (Cu Kα radiation) at (16.1±0.2)° 2θ.

28. The compound of claim 27 characterized by an X-ray powder diffraction pattern (Cu Kα radiation) substantially similar to that of FIG. 4.

29. A method of preparing the compound of claim 1 comprising slurrying (2R)-2-(4-chlorophenyl)-2-[3-(trifluoromethyl)phenoxy]acetic acid with 2-amino-2-(hydroxymethyl)propane-1,3-diol in heptane.

30. A method of preparing the compound of claim 1 comprising adding (2R)-2-(4-chlorophenyl)-2-[3-(trifluoromethyl)phenoxy]acetic acid to a solution of 2-amino-2-(hydroxymethyl)propane-1,3-diol in diisopropyl ether or methyl tert-butyl ether.

31. A method of preparing the compound of claim 1 comprising adding 2-amino-2-(hydroxymethyl)propane-1,3-diol to a solution of (2R)-2-(4-chlorophenyl)-2-[3-(trifluoromethyl)phenoxy]acetic acid in isopropyl alcohol.

32. The method of claim 31 further comprising concentrating under reduced pressure, adding heptane, and concentrating under reduced pressure.

33. A solid pharmaceutical formulation comprising the compound of claim 1 and a pharmaceutically acceptable excipient.

34. A method of treating a condition for which administration of arhalofenate, or of (2R)-2-(4-chlorophenyl)-2-[3-(trifluoromethyl)phenoxy]acetic acid or a salt thereof, is indicated, comprising administration of a therapeutically effective amount of the compound of claim 1.
